(19) United States
(12) Patent Application Publication
(10) Pub. No.: US 2003/0069644 A1
(43) Pub. Date: Apr. 10, 2003

(54) Dual-tray teletibial implant

(76) Inventors: Nebojsa Kovacevic, Plymouth, MN (US); Robbin Roberts, Plymouth, MN (US)

Correspondence Address: Steven M. Koehler, WESTMAN CHAMPLIN & KELLY, International Centre - Suite 1600, 900 South Second Avenue, Minneapolis, MN, 55402-3319, US

(21) Appl. No.: 09/972,074

(22) Filed: Oct. 5, 2001

(51) Int. Cl. A61F 2/30

(52) U.S. Cl. 623/20.32; 623/18.11; 606/102

(57) ABSTRACT

A teletibial implant is provided for measuring forces between a femur having first and second condylar surfaces and a tibia when a joint is articulated. The implant includes a medial tibial insert engaging the first condylar surface and a lateral tibial insert engaging the second condylar surface. A transducer includes a medial plate coupled to the medial tibial insert, a lateral plate coupled to the lateral tibial insert, and a bottom plate supporting the medial and lateral plates. The medial and lateral plates receive forces from the medial and lateral inserts, respectively. The bottom plate also has a plurality of spaced apart force sensors for measuring forces exerted on the medial and lateral plates.

### BACKGROUND OF THE INVENTION

The present invention pertains generally to a joint prosthesis and, more particularly, to a system that measures forces on a joint prosthesis to determine proper implantation of the prosthesis on a patient.

The human knee is the single largest joint of the human body, but due to its structure, is arguably the most vulnerable to damage. The leg consists principally of a lower bone called a tibia and an upper bone known as a femur. The tibia and femur are hinged together at the knee joint. The knee joint includes femoral condyles supported in an engagement with crescentic fibrocartilages that are positioned on the upper end of the tibia and receive the femur. The joint is held together by numerous ligaments, muscles and tendons. The patella is a similarly supported bone positioned in front of the knee joint and acts as a shield for it.

When the knee joint has been severely damaged from accident, wear, or disease, partial or total knee replacement may be the only viable solution. One type of knee replacement is shown in U.S. Pat. No. 4,340,978 issued to Buechel et al. In this patent, the tibia is resected to form a flat, horizontal platform known as tibial plateau. The amount of bone structure removed corresponding to the severity of damage to the joint and the necessary allowance needed for the prosthesis. A tibial platform is secured to the tibial plateau with posts or anchors fixed normal or perpendicular to the tibia plateau. The anchors provide additional support to the tibial platform when the joint is subjected to shear, tipping and torque forces present under normal knee articulation.

A femoral component, comprising a curved convex semi-spherical shell, covers the femoral condyles and slidably engages a concave tibial bearing insert. On a side opposite the femoral component, the tibial insert is substantially flat and slidably engages the tibial platform. Interaction of opposing surfaces of these three elements, the femoral component, the tibial insert and the tibial platform allows the prosthesis to function in a manner equivalent to a natural knee joint.

Another tibial platform and a surgical procedure for implantation is described in U.S. Pat. No. 4,822,362 issued to Walker et al.

Crucial to either the complete joint of Buechel et al. or the tibial platform of Walker et al. is proper alignment of the tibial platform on the tibial plateau. Without proper alignment, neither will function correctly whereby uneven forces on the prosthesis may result in excessive contact stresses leading to deformation and/or early wear and thus undesirable short prosthetic life.

Template assemblies have been used in implantation surgical procedures to resect the tibia and align the tibial platform. One such assembly is disclosed in U.S. Pat. No. 4,211,228 issued to Cloutier. This assembly comprises a Y-shaped handle having two flat prongs that are used to check the planes of the resected tibia for overall flatness and to hold temporarily the tibia inserts. An alignment rod, fixed to the flat handle, is aligned visually along the long axis of the tibia, as viewed laterally and anteriorally, to ensure correct positioning of the tibial platform onto the patient's tibia. Since tibial platform alignment does not include movement of the prosthetic components in order to access force loads on the joint, alignment of the tibial platform may not be optimum, realizing pressure differences across the surface of the platform which under normal articulation of the joint may cause fatigue in the prosthesis.

Developments have been made for a system to dynamically measure and analyze forces present on components of a knee joint prosthesis and all other types of prostheses. One such system is described in U.S. Pat. No. 5,197,488. The system measures forces throughout the normal range of motion of the joint using a first member attached to an outer surface of a first bone and a second member attached to an outer surface of a second bone. A transducer is located between the first and second member to measure forces thereon. However, this system does not provide isolated quantitative indications of forces present on the medial and lateral portions of the tibia. Thus, a system is needed to provide indications of forces in specific sections of the prostheses, including the medial and lateral portions of the tibia.

### SUMMARY OF THE INVENTION

A teletibial implant is provided for measuring forces between a femur having first and second condylar surfaces and a tibia when a joint is articulated. The implant has a medial tibial insert engaging the first condylar surface and a lateral tibial insert engaging the second condylar surface. A transducer includes a medial plate coupled to the medial tibial insert, a lateral plate coupled to the lateral tibial insert, and a bottom plate supporting the medial and lateral plates. The medial and lateral plates receive forces from the medial and tibial insert, respectively. The bottom plate has a plurality of spaced apart force sensors for measuring forces exerted on the medial and lateral plates.

In another aspect of the present invention, a system is provided for measuring forces applied to a joint prosthesis and adapted to be located between a first bone and a second bone that form an articulation joint. The system includes a first member attached to an outer surface of the first bone and a second member attached to an outer surface of the second bone. A transducer is positioned between the first member and the second member. The transducer has a first plate receiving forces exerted between the first bone and the second bone, a second plate receiving forces exerted between the first bone and the second bone, and a bottom plate supporting the first and second plates. The bottom plate further has a plurality of spaced apart force sensors for measuring forces exerted on the first and second plates.

### BRIEF DESCRIPTION OF THE DRAWINGS

FIG. 1 illustrates a front view of a knee prostheses according to the present invention.

FIG. 2 illustrates a rear bottom perspective view of a tibial component.

FIG. 3 illustrates a rear bottom perspective view of the tibial component of FIG. 2 with some elements illustrated in dashed lines.

FIG. 4 is a top plan view of a transducer according to the present invention.

FIG. 5 is a bottom perspective view of the transducer in FIG. 2.

FIG. 6 is a bottom plan view of the transducer in FIG. 2.

FIG. 7 is a sectional view of the transducer taken along the line 7-7 in FIG. 6.

FIG. 8 is a bottom plan view of the transducer in FIG. 2 with sensing elements.

FIG. 9 is a front elevational view of a lower portion of the knee prosthesis shown in FIG. 1.

FIG. 10 is a top perspective view of the lower portion shown in FIG. 9.

FIG. 11 is a side view of the lower portion illustrated in FIG. 9.

FIG. 12 is an exemplary environment for transmitting force signals.

### DETAILED DESCRIPTION OF THE PREFERRED EMBODIMENTS

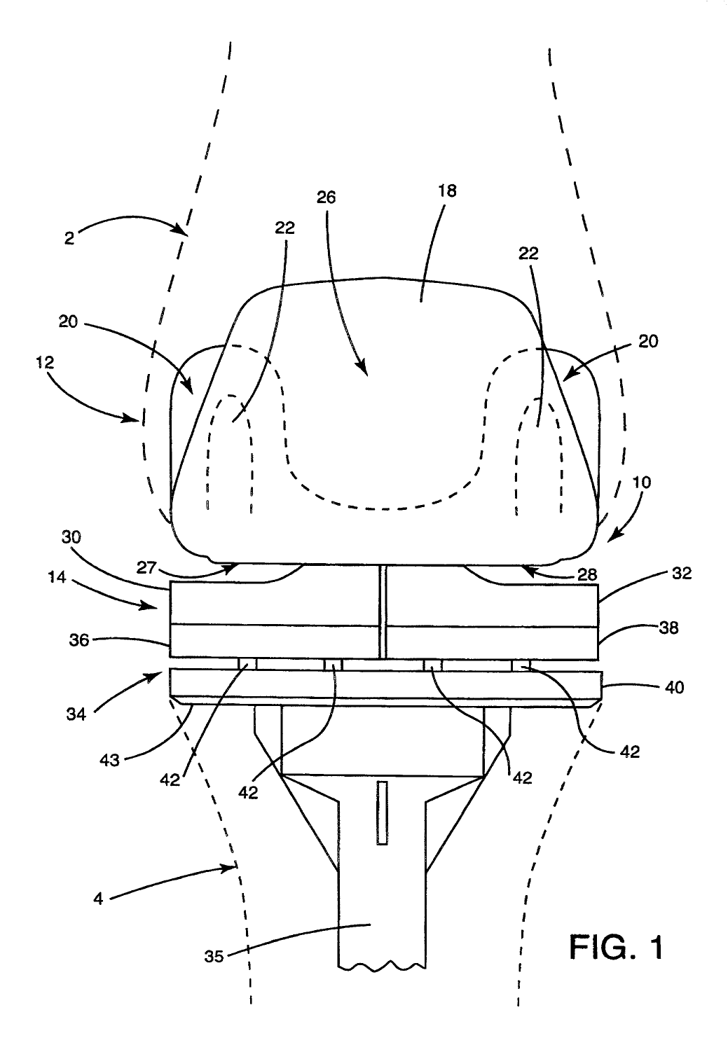

An exemplary prosthetic according to the present invention will now be described. Generally, a prosthetic includes a component mounted to the femur 2 and another component mounted to the tibia 4. Both femur 2 and tibia 4 are shown in dotted lines in FIG. 1. Measuring forces between the components aid in aligning the components properly and analyzing forces exerted on the components.

FIG. 1 further illustrates assembly 10 in accordance with an exemplary embodiment of the present invention. Assembly 10 includes femoral component 12 mounted to the femur 2 and tibial component 14 mounted to the tibia 4. Femoral component 12 includes flange 18 formed integrally with two condyles 20. Femoral component 12 includes fixing posts or anchors 22 integrally formed on femoral component 12. Posts 22 are used to fix the femoral component 12 to femur 2.

An outside surface 26 of flange 18 provides most of the bearing surface for a patella, not shown, which cooperates with femur 2 to protect the joint. Condyles 20 are provided for replacing the condylar surfaces of femur 2 and include medial condylar surface 27 and lateral condylar surface 28.

Tibial component 14 includes tibial inserts 30 and 32, transducer 34 and lower portion 35. Lower portion 35 is secured to tibia 4 and may be a solid or a hollow construction. Medial tibial insert 30 is adapted to engage medial condylar surface 27. Lateral tibial insert 32 engages lateral condylar surface 28. The medial and lateral condylar surfaces 27 and 28 exert force on medial tibial insert 30 and lateral tibial insert 32, respectively. Medial and lateral inserts 30 and 32 can be made from polyethylene or any other suitable material. In turn, inserts 30 and 32 exert forces on transducer 34. Transducer 34 includes medial plate 36, lateral plate 38 and lower plate 40. Support posts 42 support the medial plate 36 and lateral plate 38. Strain gauges (described below) are mounted directly below support post 42 and sense strain therein. When installed as a replacement assembly for a natural human knee joint, assembly 10 provides quantitative feedback in force load balance across the tibial-femoral joint.

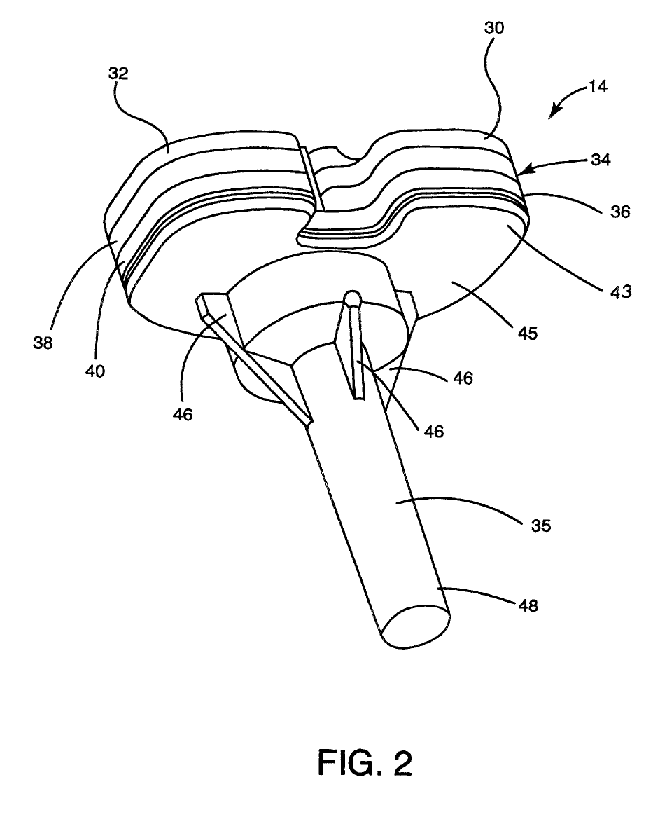

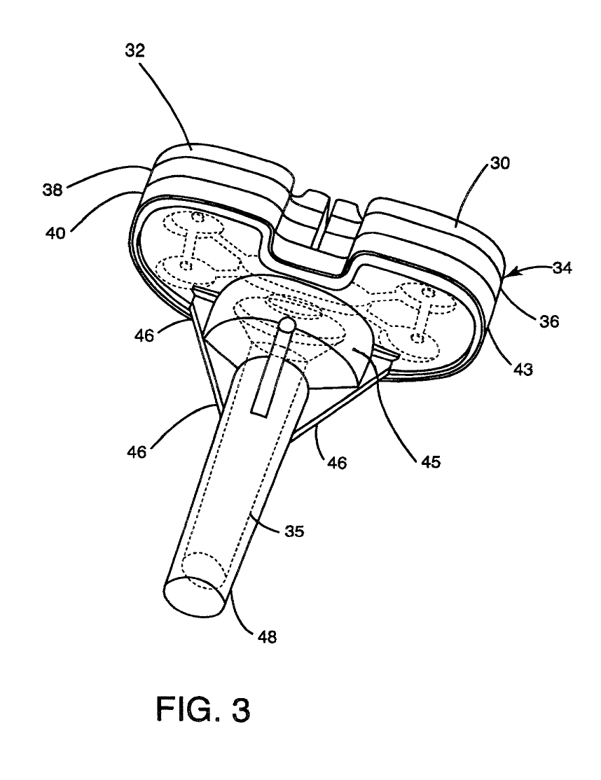

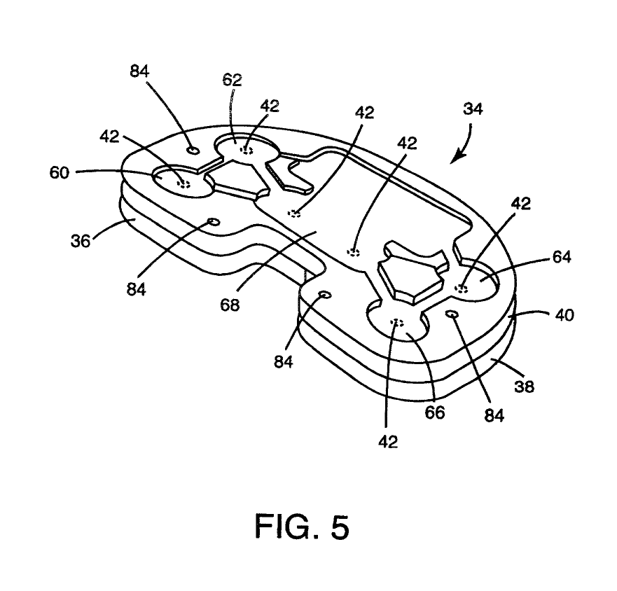

FIGS. 2-3 illustrated rear, bottom perspective views of tibial component 14. Lower portion 35 is mounted to transducer 34 with cover plate 43. Cover plate 43 protects transducer 34 from entry of unwanted elements. Cylindrical portion 45 forms a pocket (described later) for storage of components connected to transducer 34. Ribs 46 support tibial component 14 and attach cover plate 43 to stem 48. Stem 48 is inserted into the tibia 4 (FIG. 1). FIG. 3 illustrates certain elements with dashed lines to illustrate the construction of transducer 34, which is described further with reference to FIG. 5.

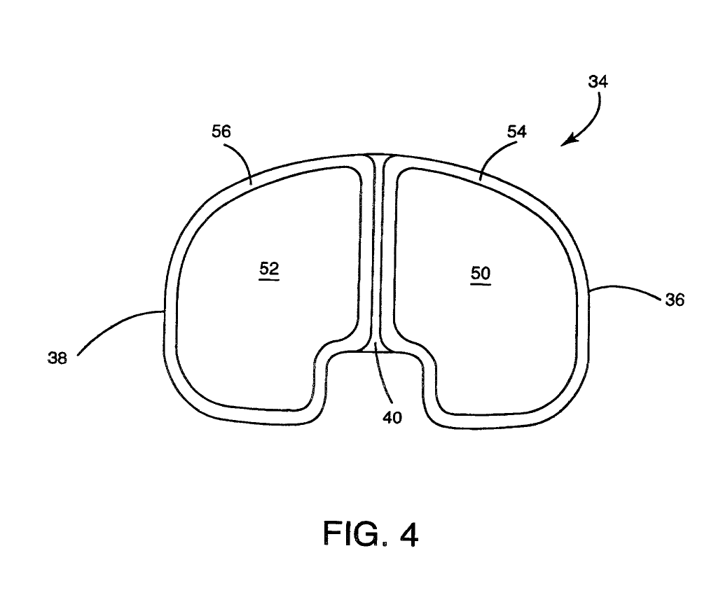

FIGS. 4-8 illustrate an exemplary embodiment of a transducer according to the present invention. Transducer 34 is symmetrically u-shaped and constructed from suitable elastic material that is responsive to forces applied to medial and lateral plates 36 and 38. Ultimately, transducer 34 is used to measure forces present on the prosthetic components. The measurements can be used to properly align the components and analyze operation of the components.

FIG. 4 illustrates a top view of transducer 34. Medial plate 36 and lateral plate 38 are spaced apart to isolate forces placed on medial and tibial inserts 30 and 32, respectively. Both medial plate 36 and lateral plate 38 include cavities 50 and 52 to receive tibial inserts 30 and 32, respectively, illustrated in FIG. 1. Walls 54 and 56 extend around the peripheral of plates 36 and 38 and define cavities 50 and 52.

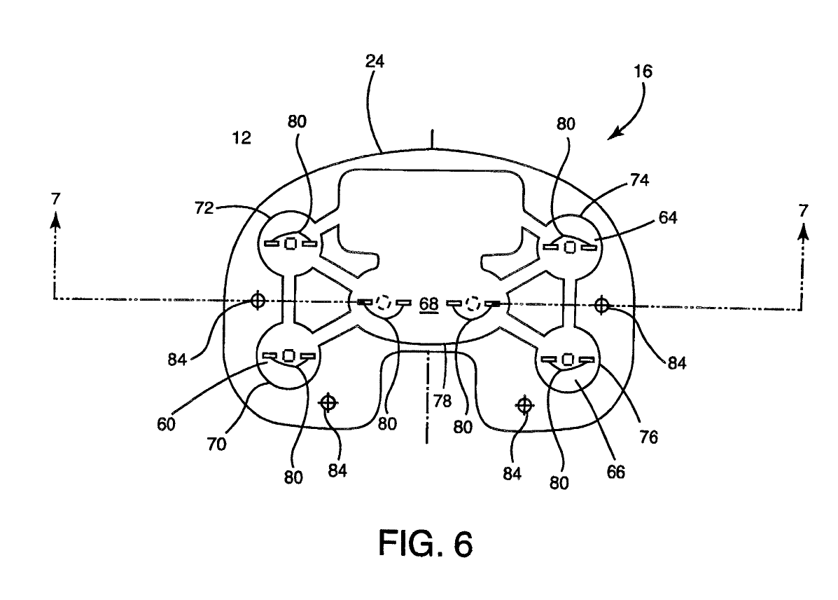

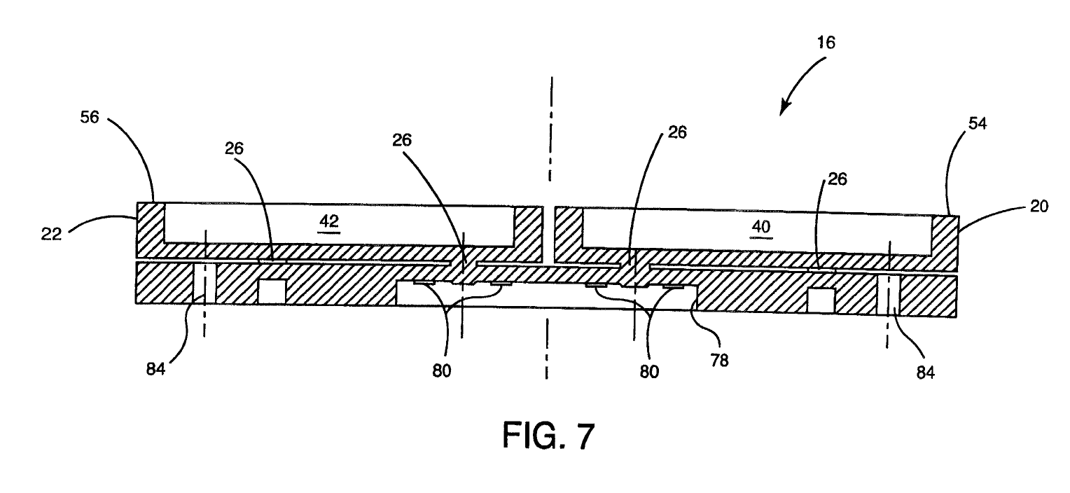

FIG. 5 illustrates a bottom perspective view of transducer 34 and FIG. 6 illustrates a bottom view of transducer 34. FIG. 7 illustrates a sectional of transducer 34 taken along line 7-7 in FIG. 6. As illustrated, lower plate 40 includes cavities 60, 62, 64, 66 and 68, which define flexures 70, 72, 74, 76 and 78, respectively. In the embodiment illustrated, cavities 60, 62, 64 and 66 are cylindrical with identical radii, while cavity 68 is elliptically shaped spanning across plates 36 and 38. Forces applied to medial and lateral plates 20 and 22 are localized and directed through support posts 42 to a corresponding flexure member. Sensor 80 measures deflection of flexures 70, 72, 74, 76 and 78. Sensor 80 can be resistive, capacitive, optical, etc. In the embodiment illustrated, a plurality of appropriate strain gauges (FIG. 4) are disposed in each respective cavity on a surface of each respective flexure member adjacent to support posts 42. Sensors 80 provide a quantitative response to forces reacted between the medial and lateral plates 36, 38 and lower plate 40, which correspond to forces carried by each of the condyles 20. Flexure 78 is unique in that it is responsive to forces from both medial and lateral plates 36 and 38. However, in order to reduce cross-talk, flexure 78 is elliptically shaped. Flexures 70, 72, 74, 76 and 78 allow forces to be measured across plate 40. In this manner, changes in forces can also be measured during articulation of the knee joint. This feature thereby allows more accurate replication of forces in a normal joint. Incorrect loading on an artificial joint can cause damage to connecting tissues such as tendons and ligaments. Apertures 84 in lower plate 40 are provided for fasteners (not shown) to secure transducer 34 to stem portion 35.

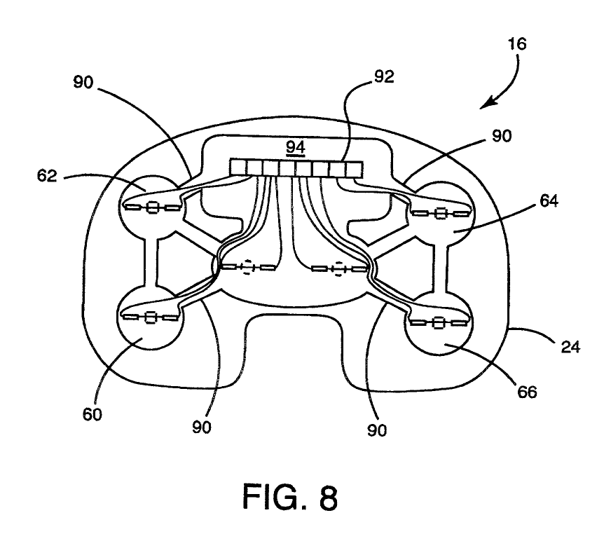

FIG. 8 illustrates a bottom plan view of transducer 34. As illustrated, channels 90 provide pathways for electrical leads from strain gauges located in cavities 60, 62, 64 and 66. All electrical leads of the strain gauges are connected to a suitable connector or terminal strip 92 placed in cavity 94. Additional leads can connect terminal strip 92 to other circuitry that will acquire transducer data, process the data and transmit the data outside the body.

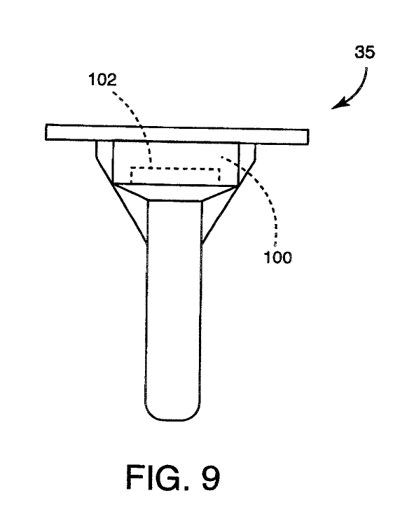

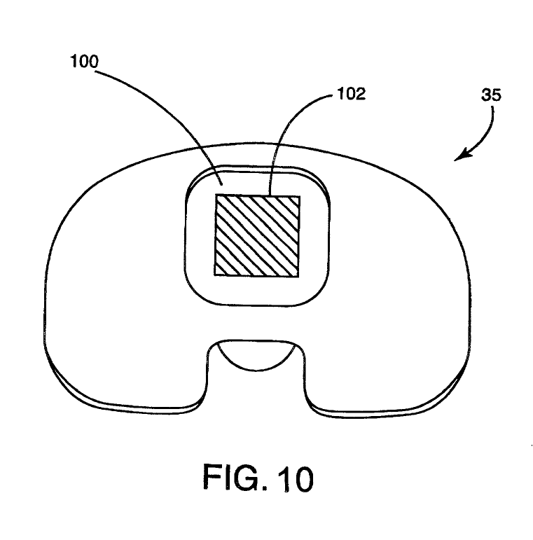

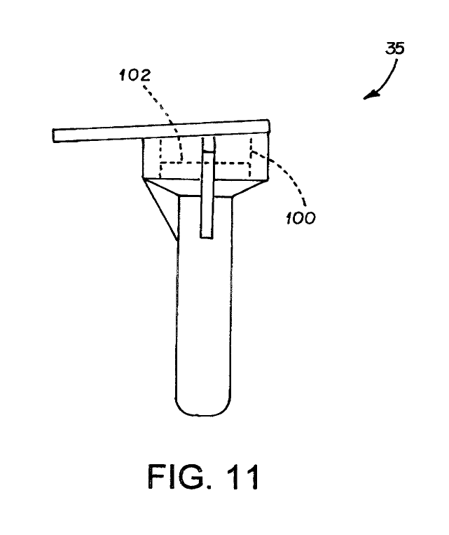

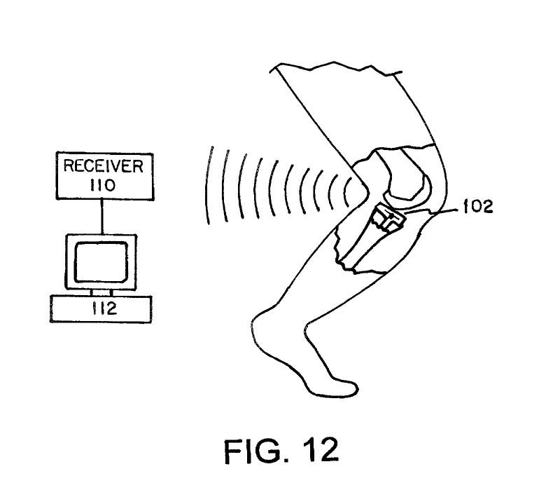

FIGS. 9-11 illustrate lower portion 35. Lower portion 35 includes pocket 100 for storage of circuitry 102. Pocket 100 opens toward transducer 34. Circuitry 102 is used to acquire, process and transmit transducer data. Circuitry 102 is couplable to terminal strip 92 of FIG. 6. As illustrated in FIG. 12, circuitry 102 can be a telemetry device that transmits signals wirelessly to a receiver 110. Location of circuitry 102 in pocket 100 of portion 35 provides an area for storage that is secure. More importantly though, the location below the transducer 34 and thus on the tibia does not interfere with operation or stability of the knee joint. Receiver 110 can then transmit signals received from telemetry device 102 to a computer 112 for further analysis.

In summary, the present invention provides an assembly and method for implantation of knee joint prostheses. The assembly accurately measures forces present on the prosthesis in vivo without cross-talk as the knee joint is articulated through partial or complete range of movements. The resulting data is collected and transmittal wirelessly for analysis to ensure proper force load distribution across the load bearing surfaces of the knee joint prosthesis. With proper load distribution, the knee joint prosthesis is optimally aligned thereby realizing increased prosthetic life.

Although the present invention has been described with reference to preferred embodiments, workers skilled in the art will recognize that changes may be made in form and detail without departing from the spirit and scope of the invention.

What is claimed is:

1. A tibial implant for measuring forces between a femur having first and second condylar surfaces and a tibia when a joint is articulated, the implant comprising:
a medial tibial insert engaging the first condylar surface;
a lateral tibial insert engaging the second condylar surface;
a transducer including:
a medial plate coupled to the medial tibial insert and receiving forces from the medial tibial insert;
a lateral plate coupled to the lateral tibial insert and receiving forces from the lateral tibial insert; and
a bottom plate supporting the medial and lateral plates and having a plurality of spaced apart flexures deflectable for forces exerted on the medial and lateral plates, the flexures including separate flexures for each of the medial and lateral plates and a common flexure for both the medial and lateral plates.

2. The implant of claim 1 and further comprising a lower portion coupled to the transducer and secured to the tibia.

3. The implant of claim 1 wherein the medial and lateral tibial inserts comprise polyethylene.

4. The implant of claim 1 wherein each flexure includes at least one of the force sensors.

5. The implant of claim 3 wherein the common flexure includes two of the force sensors.

6. The implant of claim 4 wherein each force sensor comprises a strain gauge attached to a surface of the flexure, the strain gauge providing the representative force output signal.

7. The implant of claim 5 wherein each strain gauge is resistive.

8. The implant of claim 6 wherein a plurality of support posts are attached to the bottom plate and support the medial and lateral plates, the support posts localizing forces applied to the medial and lateral plates from the medial and tibial inserts onto each flexure.

9. The implant of claim 1 and further comprising a tibial stem portion securable to the transducer and circuitry coupled to the transducer, wherein the circuitry is disposed below the transducer in the tibial stem portion and receives the representative force output signals from each force sensor.

10. The implant of claim 8 wherein the circuitry wirelessly transmits the representative force output signals outside the body.

11. A system for measuring forces applied to a joint prosthesis and adapted to be located between a first bone and a second bone that form an articulation joint, the system comprising:
a first member attached to an outer surface of the first bone;
a second member attached to an outer surface of the second bone; and
a transducer positioned between the first member and the second member, comprising:
a first plate receiving forces exerted between the first bone and the second bone;
a second plate receiving forces exerted between the first bone and the second bone; and
a bottom plate supporting the first and second plates and having a plurality of spaced apart force sensors for measuring forces exerted on the first and second plates.

12. The system of claim 11 wherein the bottom plate comprises a plurality of integrally formed flexures, wherein each flexure includes at least one force sensor.

13. The system of claim 11 wherein each force sensor comprises a strain gauge attached to a surface of the flexure, the strain gauges providing the representative force output signal.

14. The system of claim 13 wherein each strain gauge is resistive.

15. The system of claim 13 wherein a plurality of support posts are attached to the bottom plate and support the first and second plates, the support posts localizing forces applied to the first and second plates onto each flexure.

16. The system of claim 11 and further comprising a controller coupled to the transducer, wherein the controller receives the representative force output signals from each force sensor.

17. The system of claim 16 wherein the controller transmits the representative force output signals outside the body.

18. A teletibial knee implant having a transducer measuring forces between a femur and a tibia when a knee joint is articulated, comprising:
a lower portion attachable to the tibia and the transducer, having:
a pocket having an opening toward the transducer.

19. The implant of claim 18, wherein the lower portion further comprises a cover plate couplable to the transducer.

20. The implant of claim 19, wherein the lower portion further comprises a stem securable to the tibia.

21. The implant of claim 20, wherein the lower portion further comprises a plurality of ribs joined to the cover plate and the stem.
